(19) United States
(12) Patent Application Publication
(10) Pub. No.: US 2021/0299202 A1
(43) Pub. Date: Sep. 30, 2021

(54) FORMULATION OF CANNABINOID COMPOUNDS

(71) Applicant: CARDINAL ADVISORY LIMITED, Toronto (CA)

(72) Inventors: Peter CHEN, Guelph (CA); Michael ROGERS, Cambridge (CA)

(21) Appl. No.: 17/259,325

(22) PCT Filed: Jul. 10, 2019
(86) PCT No.: PCT/CA2019/050948

§ 371 Date: Jan. 11, 2021

Related U.S. Application Data
(60) Provisional application No. 62/696,003, filed on Jul. 10, 2018.

(51) Int. Cl. A61K 36/185; A61K 47/36; A61K 47/44; A61K 9/14; A61K 9/107

(52) U.S. Cl. CPC A61K 36/185; A61K 47/36; A61K 47/44; A61K 31/05; A61K 9/107; A61K 2236/333; A61K 2236/51; A61K 9/14

(57) ABSTRACT

Cannabinoid compounds are formulated with a carbohydrate, including mono-, di-, oligo-, and polysaccharides into a stable, soluble and largely flavorless powder, sugar crystals or emulsions.

### FIELD OF THE INVENTION

The present invention relates to formulations of cannabinoid compounds comprising a carbohydrate.

### BACKGROUND

The use of both medical and recreational cannabis in North America is rapidly evolving largely due to changes in marijuana laws and policies. In many jurisdictions, cannabis has been decriminalized, thus the possession and use of small personal amounts does not lead to criminal charges.

The main psychoactive compound in cannabis, THC, was first discovered in 1964 and the detection of an endocannabinoid system in mammals only occurred within the last 15 years. THC is largely responsible for the pharmacological and medicinal benefits of the marijuana plant. Other, potentially synergistic phytochemicals in cannabis include cannabinoids such as the acid metabolite THC-COOH, the non-psychotropic CBD and cannabinol (CBN), terpenes, flavonoids, and several cannabinoid analogues and modulators of the endogenous cannabinoid system. Physiological response to cannabinoids is mediated by activation of G-protein-coupled cannabinoid receptors (GPCR) in the brain and peripheral tissues. Specifically, cannabinoids act as inverse agonist ligands to GPCR. THC is both thermolabile and photolabile, therefore extended storage of THC and/or cannabis can lead to the oxidation of THC to the non-psychoactive metabolite, CBN.

Cannabis may be prescribed in the form of medicinal marijuana and is intended as a physician-recommended herbal therapy. The natural drug is typically smoked or ingested orally, as these methods have been proven to be most effective. When smoked, pulmonary absorption of the released THC causes immediate psychotropic effects beginning within seconds to minutes, reach a peak plasma THC concentration in roughly 6-10 minutes and level off within 2-3 hours. Oral ingestion follows a more delayed and unpredictable response, generally resulting in psychotropic effects starting 30-90 minutes after ingestion and reach a peak plasma concentration in 2-6 hours. The effects may last as long as 4-12 hours with oral administration, depending on dosage.

Although smoking of the dried cannabis plant matter can lead to a fast psychotropic response and can simultaneously decarboxylate inactive cannabinoid acids to their active phenolic form, inhalation of cannabis smoke suffers from the same health concerns related to tobacco smoking. Oral administration offers several advantages including increased pulmonary health and more precise dosing of specific active ingredients such as THC and CBD. Oral administration does, however, suffer certain shortcomings and challenges as well. In acidic conditions, such as that of the stomach, THC can rapidly degrade to form isomerized metabolites, 11-OH-THC and THC-COOH. Extensive liver metabolism further reduces the oral bioavailability of THC to roughly 2-14%, while inhalation of marijuana smoke offers a systemic bioavailability generally ranging between 10-35%.

Alternative cannabis formulations are available. Sativex™ is an oromucosal spray made from a whole-plant cannabis extract, propylene glycol, peppermint oil and ethanol, and is approved for use in Canada to treat neuropathic pain associated with multiple sclerosis and for palliative care for patients with cancer pain as an alternative to opiates. Similarly, certain licensed producers in Canada can produce and distribute cannabis oils for sublingual administration or in capsular form. Cannabis oil consists of whole-plant extracts diluted using carrier oils typically consisting of medium chain triglycerides (MCT).

Health conscience consumers and patients may favor cannabis-infused edible products, including beverages. However, the hydrophobic nature of cannabis extracts and its main active components, THC and CBD, pose complications in formulating such products. Cannabinoid plant extracts are typically a resinous sticky oil with an amorphous nature that undergoes degradation through different mechanisms resulting in a low oral bioavailability. Directly incorporating cannabis extract or cannabis oil into a food or beverage can lead to low bioavailability and unpredictable dosing. In an aqueous solution, phase separation can occur between the aqueous phase and the oil phase leading to incomplete administration of an exact dose of cannabinoid. Moreover, the flavor imparted by the terpene content can negatively affect sensory properties of the food or beverage product. Lastly, the lack of crystal lattice structure makes cannabinoids susceptible to oxidative degradation.

Therefore, there is a need in the art for novel formulations of cannabinoid compounds which may mitigate some or all of the problems of the prior art.

### SUMMARY OF THE INVENTION

In general terms, the invention comprises cannabinoid formulations comprising one or more carbohydrate excipients, which may mitigate the labile properties of cannabinoids, and/or increase water solubility, and/or improve bioavailability, and/or response time for psychotropic or other physiological effects. These formulations may provide products which provide 1) an alternative to smoking cannabis; 2) a product that delivers an accurate dose of THC and CBD when ingested orally; 3) a flexible food-grade ingredient for use in beverages and baked food products; and/or 4) a product that has improved water solubility properties so as to enhance oral bioavailability.

The cannabis formulations of the present invention may be crystallized or powdered depending on the type of carbohydrates used, and may be incorporated, for example, in beverages, such as smoothies or milkshakes, and foods, such as baked products. The final product can be infused with a wide range of THC and/or CBD concentrations and may provide the ability to measure a precise dose by use of standard household measuring devices (teaspoon, tablespoon, cup). The high water solubility of some embodiments may also enhance oral bioavailability.

In some aspects, the formulation may comprise a cannabis emulsion in water that is physically blended, such as by using a high speed dispersion homogenizer. This creates a cannabis-infused liquid which can be directly consumed or mixed into a food or beverage.

Without restriction to a theory, in some embodiments, it is believed that the carbohydrate ingredient(s) and the cannabinoids combine into a stable and water-soluble structure which does not involve chemical bonding, and may involve adsorption or other physical interaction with a structural crystal or other physical structure of the carbohydrate.

In one aspect, the invention comprises a cannabinoid formulation comprising a cannabinoid extract, a monosaccharide or a disaccharide, and optionally, a lipid. Suitable mono- or disaccharides may comprise fructose, glucose, galactose, sucrose, maltose, isomaltose, or lactose, natural or synthetic derivatives thereof, or combinations thereof. In some embodiments, particularly where an optional lipid is incorporated, the formulation may include a solubilizer or emulsifier, such as polymeric solubilizers well known as drug excipients (Soluplus™), HLB 7, or soy lecithin. Suitable lipids may include a medium chain triglyceride oil (MCT), or a plant oil, such as palm, olive, canola, avocado, hemp seed, or grape seed oil.

In another aspect, the invention comprises a cannabinoid formulation comprising an oligo- or polysaccharide, but which does not include a lipid. Suitable oligo- or polysaccharides may include water soluble complex carbohydrates, such as a starch, a polyol or sugar alcohol, maltodextrin, cellulose or cellulose derivatives such as hydroxypropyl methylcellulose (HPMC), hydroxyethyl methyl cellulose (HEMC), hydroxypropyl methylcellulose acetate succinate (HPMCAS), sodium or calcium alginate, acacia gum, xantham gum, guar gum, or combinations thereof. In some alternative embodiments, the polysaccharide may comprise cellulose or cellulose derivatives which are not water soluble below 25° C. Optionally, an emulsifier and/or stabilizer may be included.

In some embodiments, the formulation may be a solid form, such as a crystallized and/or powdered particular form, or a liquid or semi-solid form.

The final product can be formulated to comprise a precise and measured THC and/or CBD concentration, and may have the advantage of allowing an exact dose to be measured using standard household measuring systems (teaspoon, tablespoon, cup). Alternatively, the final product can be packaged and dispensed in single dosage forms, having a known precise dosage, such as a tablet, capsule, or other single edible article, which may be a food or beverage.

The formulation may include food additives, such as flavoring agents, anti-oxidants, food stabilizers or preservatives, known for their advantageous properties in food processing.

Other features and advantages of the present disclosure will become apparent from the following detailed description. It should be understood, however, that the detailed description and the specific examples while indicating preferred embodiments of the disclosure are given by way of illustration only, since various changes and modifications within the spirit and scope of the disclosure will become apparent to those skilled in the art from this detailed description.

### BRIEF DESCRIPTION OF THE DRAWINGS

Exemplary embodiments of the invention may be described below in reference to the attached drawings.

FIG. 1 shows a flowchart of a scheme for formulating cannabinoids with sucrose.

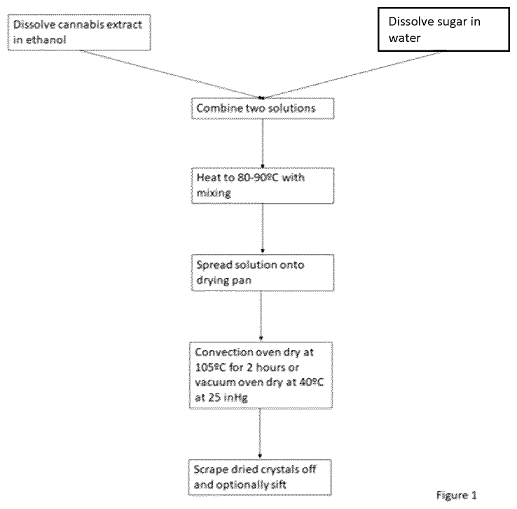

FIG. 2 shows a flowchart of an alternative scheme for formulating cannabinoids with sucrose.

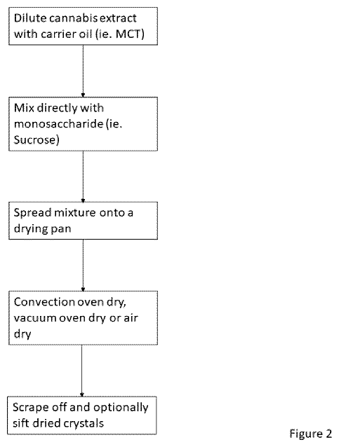

FIG. 3 shows a flowchart of a scheme for formulating cannabinoids with a polysaccharide to produce a powder.

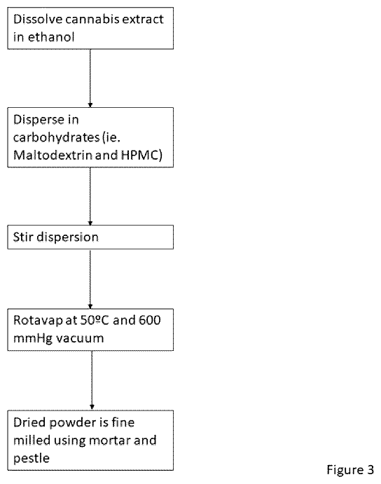

FIG. 4 shows a flowchart of a scheme to produce a cannabinoid simple sugar emulsion.

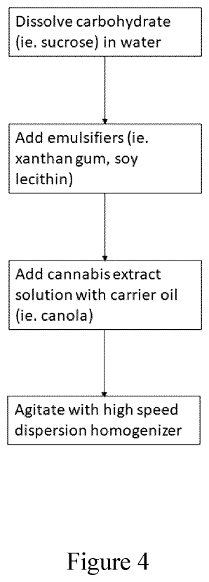

FIG. 5 shows a flowchart of an alternative scheme to produce a cannabinoid simple sugar emulsion.

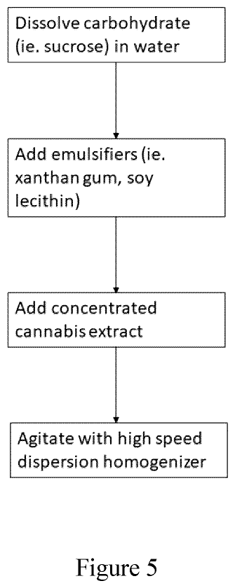

### DETAILED DESCRIPTION

The present invention comprises formulations of cannabinoid compounds and a carbohydrate, and methods of producing such formulations.

Cannabinoids are compounds which act on cannabinoid receptors in cells, which can alter neurotransmitter release in the brain. Cannabinoids were originally found in Cannabis sativa L., the origin of marijuana and hashish. Marijuana or its components have been reported in the scientific literature to alleviate the symptoms of a broad range of conditions including multiple sclerosis and forms of muscular spasm, including uterine and bowel cramps; movement disorders; pain, including migraine headache; glaucoma, asthma, inflammation, insomnia, and high blood pressure. There may also be utility for cannabinoids as an oxytoxic, anxiolytic, anti-convulsive, anti-depressant and/or anti-psychotic agent, or anti-cancer agent, as well as an appetite stimulant.

Many chemically related compounds, collectively classified as cannabinoids, have been isolated from Cannabis sativa L., Cannabis indica and Cannabis ruderalis. The cannabinoids usually divided in the groups of classical cannabinoids, non-classical cannabinoids, aminoalkylindole derivatives and eicosanoids. Classical cannabinoids are isolated from Cannabis sativa L. or they can comprise synthetic analogs of these compounds. Non-classical cannabinoids are bi- or tricyclic analogs of tetrahydrocannabinol (THC) while aminoalkylindoles form a group which differs structurally substantially from classical and non-classical cannabinoids.

In various embodiments, the cannabinoid can include, but is not limited to, cannabinoid compounds that may naturally occur in different combinations and relative quantities in the plant tissues of various species, subspecies, hybrids, strains, chemovars, and other genetic variants of the genus Cannabis, including material that may variously be classified as “marijuana” and “hemp” in accordance with various legal or technical definitions and standards.

An exemplary cannabinoid comprises THC, having the formula (I):

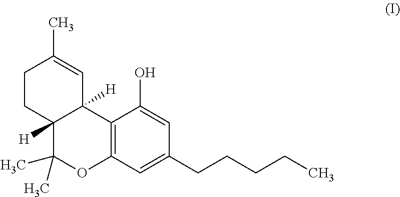 [SMILES: CCCCCc1cc(O)c2c(c1)OC(C)(C)[C@@H]1CCC(C)=C[C@@H]21]

which includes delta-9-tetrahydrocannabinol (D9THC), acknowledged to be the main psychoactive compound in Cannabis.

Cannabidiol (CBD) IUPAC: 2-[(1R,6R)-6-isopropenyl-3-methylcyclohex-2-en-1-yl]-5-pentylbenzene-1,3-diol, having the formula (II):

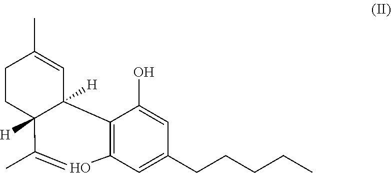

is another cannabinoid which may be present in embodiments of the present invention. Although CBD is not known to have the psychotropic effects of THC, it is still considered to have a wide scope of potential medical and therapeutic applications. CBD may be derived from industrial hemp which has negligible amounts of THC, and may be legally grown and consumed in Canada and the United States.

The cannabinoid component may also include various other cannibinoids such as tetrahydrocannabinolic acid (THCA), delta-8-tetrahydrocannabinol (D8THC),), cannabidiolic acid (CBDA), cannabinol (CBN), cannabinolic acid (CBNA), tetrahydrocannabinovarin (THCV), tetrahydrocannabinovarinic acid (THCVA), cannabidivarin (CBDV), cannabidivarin acid (CBDVA), cannabigerol (CBG), cannabigerolic acid (CBGA), cannabichromene (CBC), cannabichromenic acid (CBCA), cannabinodiol (CBND), and cannabinodiolic acid (CBNDA).

In one aspect, the invention uses cannabinoids present in a cannabis extract produced from cannabis plant material, preferably produced by a selective extraction process. The term “selective” as used herein in reference to a solvent, a solid phase, chromatographic or separation system of a solvent or solid phase that selectively extracts or purifies a target substance with greater specificity, relative to another, different substance. In some embodiments, the selective system purifies the target substance such that the extract has at least 2 fold, 3 fold, or 5 fold greater concentration of a target substance than in the original composition. Preferably, the concentration of non-target substances is reduced at the same time.

Suitable cannabis extracts are commercially available or may be produced using methods well known to those skilled in the art and need not be described herein. One suitable selective extraction method is described in co-owned and co-pending application PCT Patent Application No. PCT/CA2018/051508 entitled “Extraction and Purification of Cannabinoid Compounds” and filed Nov. 27, 2018, the entire contents of which are incorporated herein by reference (where permitted).

Cannabis extracts tend to be resinous and oily. Therefore, the cannabinoid extract may be provided in a liquid carrier. For example, the cannabinoids may be selectively extracted in an alcoholic solution, such as ethanol or an ethanol solution, which may be a 40-98% ethanol in water, and used directly to create the formulations of the present invention. Alternatively, if the extract is in a different form, such as a resin or an oil, it may be dissolved in an alcohol, such as ethanol. Ethanol is preferred as it dissolves cannabinoid compounds, is miscible with water, and is potable. The liquid carrier may also comprise a carrier lipid, such as a vegetable oil or MCT oil, or a mixture of an alcohol such as ethanol and a carrier lipid.

In all cases, it is preferred that all components are suitable for human consumption.

### Simple Sugar Formulations

Accordingly, in one aspect, the invention comprises a water-soluble cannabinoid formulation comprising a simple sugar. As used herein, a “simple sugar” means a mono- or disaccharide, such as glucose, dextrose, fructose, sucrose, lactose, maltose, isomaltose and the like. The resulting formulation may comprises a solid particulate form, such as a powder, a liquid syrup or elixir, which may be an emulsion.

In some embodiments, the formulation comprises a simple sugar to cannabinoid ratio of between about 90:10 to about 98:2, by weight. In a preferred embodiment, the ratio may be about 95:5. For example, a 1 g sample may comprise about 50 mg of cannabinoid and about 950 mg of simple sugar. This preferred ratio has been found to produce solid sugar crystals with a suitable physical consistency while being able to deliver a substantial dose of cannabinoid compounds.

In some embodiments, the formulation may be produced by a method which comprises the steps of:
a) producing a cannabinoid extract in a liquid carrier;
b) producing an aqueous solution of the simple sugar; and
c) mixing the liquid carrier and the sugar solution.
   The mixture may be optionally heated, for example at about 80° to about 90° C., to facilitate the mixing process.

The liquid carrier may comprise ethanol, or an ethanol solution. In some embodiments, the cannabis extract may comprise an extract in an oil or resin, and may diluted with a liquid carrier such as MCT, palm, olive, canola, avocado, hemp seed, or grape seed oil, so the doses are more palatable, as concentrated cannabis extract can contain up to 700 to 900 mg THC/g. A solubilizer, stabilizer and/or emulsifier may be added. Various additives are known to promote solubility of hydrophobic compounds into water, or initiate or stabililize emulsions. They may be referred to herein as emulsifiers or stabilizers, and include acacia gum, guar gum, soy lecithin, or xanthan gum. Suitable solubilizers may include Soluplus™ (polyvinyl caprolactam-polyvinyl acetate-polyethylene glycol graft copolymer (PCL-PVAc-PEG)), HLB 7, polyoxyethylene sorbates (e.g. Tween® 20 and Tween® 80), polyoxyl hydrogenated castor oils (e.g. Cremphor® EL and Cremophor® RH 40), Tyloxapol®, polyoxyethyelene ethers (Brij® series) and alkoxylated fatty acid esters (Myrj® series), sorbitan esters (Span® series) and others known to a person skilled in the art. The solubilizers may be used alone or in combination, or in combination with an emulsifier, and/or include a surfactant.

The proportion of cannabis extract to simple sugar may be varied. It is preferred to add no more oil than necessary to maintain a sugar crystal that is not overly soft and still flows as a powder.

In some embodiments, the product is dried to a solid form, for example by air-drying or drying in a vacuum or oven. The dried product may be crushed, ground, milled or sifted to form a powder.

In other embodiments, the product may comprise a liquid emulsion of the cannabinoids in a lipid carrier and an aqueous solution of a simple sugar. In this case, an emulsifier may be included in one or both of the cannabinoid liquid carrier or sugar solution, and the mixture may be emulsified in a homogenizer. An emulsion is a system consisting of two immiscible liquid phases (oil and water), one of which is dispersed throughout the other as fine droplets, the system being stabilized by a third component, the emulsifying agent. Emulsions are inherently unstable, and emulsifiers are essential for both their initial formation and long-term stability. Emulsions may be oil in water (oil phase dispersed in the aqueous phase) or water in oil (water phase dispersed in the oil phase) emulsions. A variety of other systems such as oil in water in oil emulsions and water in oil in water emulsions are also known in the art. Several emulsion stabilizers or emulsifying agents are known in the art and include surfactants and phospholipids. Examples of surfactant emulsifiers include polyoxyethylene sorbates (e.g. Tween® 20 and Tween® 80), polyoxyl hydrogenated castor oils (e.g. Cremphor® EL and Cremophor® RH 40), Tyloxapol®, polyoxyethyelene ethers (Brij® series) and alkoxylated fatty acid esters (Myrj® series), sorbitan esters (Span® series) and others know to a person skilled in the art. Examples of phospholipids that may be used as emulsion stabilizers include phospholipids (e.g. phosphatidylcholine, phosphatidylinositol, phosphatidylglycerol).

High speed homogenizers for producing oil-water emulsions are well known. Any effective combination of agitation speed or blending temperature for a full cannabis-water emulsion may be used. Stock solutions can be any combination of carbohydrates and emulsifiers with any concentration of cannabis extract with carrier oil. Concentrations of THC/CBD can be modified depending on volume of liquid produced.

### Poly- and Oligosaccharide Formulations

In another aspect, the invention may comprise a cannabinoid formulation comprising an oligosaccharide or a polysaccharide. Oligosaccharides comprise a short polymer of 3 to 10 monosaccharide units, and may include fructooligosaccharides (FOS), galactooligosaccharides (GOS), xylooligosaccharide (XOS), isomaltooligosaccharides (IMO) such as isomaltose, panose, isomaltotriose, isomaltotetraose, isomaltopentaose, nigerose, kojibiose, and higher branched oligosaccharides. Suitable polysaccharides may include water soluble cellulose derivatives such as hydroxypropylmethyl cellulose (HPMC), hydroxyethylmethyl cellulose (HEMC), maltodextrin, acacia gum, xantham gum, guar gum, or combinations thereof. In some embodiments, a combination of oligo- and/or polysaccharides may be preferred. For example, a mixture of maltodextrin and a binder such as HPMC may be used. In another example, a mixture of maltodextrin and a stabilizer such as acacia gum may be used.

In some embodiments, the formulation does not include any substantial amount of a lipid, or any hydrophobic component other than the cannibinoids themselves.

In some embodiments, the polysaccharide may comprise cellulose and cellulose derivatives which are not water-soluble, such as microcrystalline cellulose (MCC) or starch which are still hydrophilic. Starch is soluble in water only upon heating. These alternative embodiments may comprise a soluble polysaccharide in addition to the non-soluble polysaccharide. The non-soluble component then functions as a solid support or carrier for the soluble and/or hydrophobic components of the formulation.

A polymeric solubilizer such as Soluplus™ (polyvinyl caprolactam-polyvinyl acetate-polyethylene glycol graft copolymer (PCL-PVAc-PEG)) may be added to enhance water solubility if necessary or desired. The solubilizer may also act as a binder.

The carbohydrate component does not directly solubilize the cannabinoid compounds such as by inclusion in a molecular cavity, such as with cyclodextrins. Therefore, in some embodiments, the formulation does not include any appreciable amounts of a cyclodextrin.

In some embodiments, the ratio of cannabinoid compounds to the oligo- or polysaccharide may be in the range of about 1:50 to about 1:200 by weight. In a preferred embodiment, a ratio of 1:5:100 of cannabinoid to HPMC to maltodextrin is produced.

The cannabis extract may be used in a liquid carrier, such as ethanol, which in some embodiments is substantially (<5% vol.) or entirely free of water. The oligo- or polysaccharide is mixed directly into the liquid carrier such that the oligo- or polysaccharide is well dispersed in the liquid carrier. The dispersion may then be dried, such as in a vacuum or vacuum oven. A heating step may be incorporated at the mixing and/or drying step, for example to about 60° to about 105° C.

The resulting product is an oligo- or polysaccharide based solid, which carries the cannabinoid compounds, and which may be used as a flowable powder.

If the final product desired is a homogenized elixir solution, the mixture of carbohydrate, water, and cannabis extract mixed with a carrier oil can be blended into an emulsion. The carbohydrate may be dispersed or dissolved in the aqueous phase.

### Formulations

The concentration of the desired cannabinoid (eg. THC and/or CBD) in the final formulation may range from about 0.1% to about 10% (by weight), but are preferably in the range of about 0.4-1.0%. The concentration can be modified depending on the potency of the initial cannabis extract. At a 1% concentration, a single 10 mg dose would require 1.2 g powder which can be dissolved in 1 cup (236.6 mL) water.

The carbohydrate-cannabinoid formulations described herein may have precisely measured doses of desired cannabinoids, as the quantity and concentration of the cannabinoid extract may be controlled. The solid or liquid formulations described herein may be incorporated into oral dosage units similar to pharmaceutical delivery vehicles, or may be incorporated into food or beverage products, including ingredients or ready-to-eat items,

### EXAMPLES

The following examples are intended to illustrate specific embodiments of the invention described herein, and not be limiting of the claimed invention in any way.

### Example 1—Simple Sugar Formulation

Cannabis extracts from a sequential ethanol extraction process is used as starting material. The extract can be kept in its dissolved form in ethanol. If the cannabis extract has been previously dried, it can be reconstituted in ethanol. The THC and CBD concentration in ethanol can range between 0.1 to 2 mg/mL. A preferred concentration for producing cannabis crystals was found at a range between 0.8 to 1.0 mg/mL. Monosaccharide such as fructose or glucose, or disaccharide such as lactose, maltose or sucrose (eg. granulated table sugar) is weighed out so as to constitute 95% of the total weight. The final product appears to crystallize very well at a 95% sugar to 5% THC/CBD ratio (wt.). At much higher THC/CBD content, the crystals were soft and/or clumped together. The sugar is dissolved separately in water at equal volume to that of cannabis extract dissolved in ethanol. The two solutions are combined and mixed. The combined solution is heated to 80-90° C. for 30 min with continuous mixing. The solution is then spread on a shallow drying pan and oven dried at 105° C. for 2 hrs or to dryness. The resulting product is a translucent thin sheet with glass-like appearance. The dried material may be scraped off as small crystalline particles.

### Example 2—Simple Sugar Powder with Carrier Oil

Cannabis extracts from a sequential ethanol extraction process is used as starting material. The extracts are combined as a concentrated resinous cannabis oil. The THC and CBD concentration in the extract can range between 700 to 900 mg/g. The resin extract is diluted with a suitable volume of a carrier oil such as MCT. A monosaccharide such as fructose or glucose, or a disaccharide such as sucrose, lactose or maltose, is weighed out so as to constitute 95% of the total weight and mixed directly with the carrier oil/extract mixture. The oil carrier adsorbs entirely into the sugar particles. Similarly to Example 1, the final product behaved optimally at the 95% sugar to 5% THC or CBD ratio (wt.).

### Example 3—Simple Sugar Syrup Emulsion

Oily or resinous cannabis extracts from a sequential ethanol extraction process is used as starting material and is mixed with a carrier oil like MCT or canola. Granulated table sugar is dissolved in water with effective quantities of soy lecithin and a stabilizer (xanthan gum or acacia gum) to create a stock syrup solution. The cannabis extract is then mixed with the syrup and blended into a physical emulsion using a high speed dispersion homogenizer at 10,000 rpm for 5 minutes over a hot plate set at 60° C., to produce a homogenous and stable cannabis-water emulsion. The THC and/or CBD concentration may be 0.1% to about 10% (by weight).

### Example 4—Polysaccharide Powder

Cannabis extracts from a sequential ethanol extraction process is used as starting material. The extract can be kept in its dissolved form in ethanol. If the cannabis extract has been previously dried, it can be reconstituted in pure ethanol. The alcoholic solution may be combined with a polysaccharide, with an optional binder. For example, maltodextrin (Dextrose Equivalent of about 1 to about 20, preferably between about 4 and 7) and HPMC binder are added to the cannabis extract in ethanol. The volume of ethanol is arbitrary so as long as it dissolves the extract completely and is sufficient to disperse the carbohydrates. A ratio of 1:5:100 CBD/THC to HPMC to tapioca maltodextrin (w/w/w) may be preferred. The dispersion is stirred and placed under rotary vacuum drying at 50° C. and 600 mmHg vacuum. The dried powder may then be ground into a fine powder using a mortar and pestle or the like. The polysaccharide likely acts as a carrier agent for the cannabinoids, which associate with the polysaccharide structure. A binder such as HPMC may aid in the formulating process.

### Example 5—Polysaccharide Powder with Stabilizer

Cannabis extract in 100% ethanol, tapioca maltodextrin (DE of between about 4 and 7) and acacia gum stabilizer are mixed in equal weight quantities, to produce a homogenous dispersion and spread on a shallow drying pan and air dried. The resulting product can be scraped off as small crystalline or powder particles and sifted for consistent fine texture.

Various cannabinoid concentrations are produced by varying the concentration of the cannabinoids in the ethanol extract.

### Definitions and Interpretation

The description of the present invention has been presented for purposes of illustration and description, but it is not intended to be exhaustive or limited to the invention in the form disclosed. Many modifications and variations will be apparent to those of ordinary skill in the art without departing from the scope and spirit of the invention. Embodiments were chosen and described in order to best explain the principles of the invention and the practical application, and to enable others of ordinary skill in the art to understand the invention for various embodiments with various modifications as are suited to the particular use contemplated. To the extent that the following description is of a specific embodiment or a particular use of the invention, it is intended to be illustrative only, and not limiting of the claimed invention.

References in the specification to “one embodiment”, “an embodiment”, etc., indicate that the embodiment described may include a particular aspect, feature, structure, or characteristic, but not every embodiment necessarily includes that aspect, feature, structure, or characteristic. Moreover, such phrases may, but do not necessarily, refer to the same embodiment referred to in other portions of the specification. Further, when a particular aspect, feature, structure, or characteristic is described in connection with an embodiment, it is within the knowledge of one skilled in the art to combine, affect or connect such aspect, feature, structure, or characteristic with other embodiments, whether or not such connection or combination is explicitly described. In other words, any element or feature may be combined with any other element or feature in different embodiments, unless there is an obvious or inherent incompatibility between the two, or it is specifically excluded.

It is further noted that the claims may be drafted to exclude any optional element. As such, this statement is intended to serve as antecedent basis for the use of exclusive terminology, such as “solely,” “only,” and the like, in connection with the recitation of claim elements or use of a “negative” limitation. The terms “preferably,” “preferred,” “prefer,” “optionally,” “may,” and similar terms are used to indicate that an item, condition or step being referred to is an optional (not required) feature of the invention.

The singular forms “a,” “an,” and “the” include the plural reference unless the context clearly dictates otherwise. The term “and/or” means any one of the items, any combination of the items, or all of the items with which this term is associated.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. A recited range (e.g., weight percents or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc.

As will also be understood by one skilled in the art, all language such as “up to”, “at least”, “greater than”, “less than”, “more than”, “or more”, and the like, include the number recited, and such terms refer to ranges that can be subsequently broken down into sub-ranges as discussed above. In the same manner, all ratios recited herein also include all sub-ratios falling within the broader ratio.

1: A cannabinoid formulation comprising:
(a) a cannabinoid or cannabinoid extract; and
(b) a mono- or disaccharide;
wherein the cannabinoid content is 2% to 10% by weight of the mono- or disaccharide.

2: The cannabinoid formulation of claim 1 wherein the cannabinoid content is about 5% by weight of the mono- or disaccharide.

3: The cannabinoid formulation of claim 1 further comprising a lipid.

4: The cannabinoid formulation of claim 3 wherein the lipid comprises MCT oil, palm oil, olive oil, canola oil, avocado oil, hemp seed oil, or grape seed oil, or combinations thereof.

5: The cannabinoid formulation of claim 1 which is dried and processed to a powder.

6: The cannabinoid formulation of claim 1 which is an oil-water emulsion comprising an emulsifier and/or stabilizer.

7: A cannabinoid formulation comprising:
(a) a cannabinoid or cannabinoid extract; and
(b) an oligo- or polysaccharide;
and which does not include a lipid nor a cyclodextrin.

8: The cannabinoid formulation of claim 7 wherein the oligo- or polysaccharide comprises one or more of a fructooligosaccharide, galactooligosaccharide, xylooligosaccharide, isomaltooligosaccharide, hydroxypropylmethyl cellulose, hydroxyethylmethyl cellulose, maltodextrin, acacia gum, xantham gum, or guar gum.

9: The cannabinoid formulation of claim 8 comprising maltodextrin and a polysaccharide binder.

10: The cannabinoid formulation of claim 9 comprising a weight ratio of cannabinoids to hydroxypropylmethyl cellulose to maltodextrin of 1:5:100.

11: The cannabinoid formulation of claim 8 comprising maltodextrin and a stabilizer.

12: The cannabinoid formulation of claim 11, comprising a weight ratio of maltodextrin to a stabilizer such as acacia gum, xanthan gum or guar gum of about 1:1.

13: The cannabinoid formulation of claim 7 comprising starch, cellulose, a cellulose derivative such as microcrystalline cellulose, or other polysaccharide which is not soluble in water at less than 25° C.

14: A method of producing a cannabinoid formulation of claim 9, comprising the steps described in any example herein.
